(19) United States
(12) Patent Application Publication
(10) Pub. No.: US 2025/0152343 A1
(43) Pub. Date: May 15, 2025

(54) TRANSCATHETER PROSTHETIC HEART VALVE DELIVERY DEVICE WITH SUPPORT EXTENSIONS AND METHOD OF LOADING A PROSTHETIC HEART VALVE TO A DELIVERY DEVICE

(71) Applicant: Medtronic, Inc., Minneapolis, MN (US)

(72) Inventors: Amirsepehr Azimian, Costa Mesa, CA (US); Christopher J. McCabe, Oranmore (IE); David M. Martin, Oranmore (IE); Wayne M. Falk, Minneapolis, MN (US)

(21) Appl. No.: 18/729,741

(22) PCT Filed: May 1, 2023
(86) PCT No.: PCT/IB2023/054515

§ 371 Date: Jul. 17, 2024

Related U.S. Application Data
(60) Provisional application No. 63/337,422, filed on May 2, 2022.

(51) Int. Cl. A61F 2/24

(52) U.S. Cl. CPC A61F 2/2418; A61F 2/2436

(57) ABSTRACT

A system including a prosthetic heart valve and a delivery device. The prosthesis includes a stent frame supporting a valve structure. The stent frame is transitionable between compressed and expanded conditions. The delivery device includes outer and inner shaft assemblies. The outer shaft assembly includes a capsule extending distally from an outer shaft. The inner shaft assembly is coaxially received within the outer shaft assembly, and includes an inner shaft, a valve retainer, and a tip unit. A proximal section of the tip unit extends from, and has an outer diameter greater than, the inner shaft. In a delivery state, the prosthesis is contained in the compressed condition over the inner shaft assembly by the capsule, a first region of the stent frame is connected to the valve retainer, and at least a portion of a second region of the stent frame contacts the proximal section of the tip unit.

### FIELD

The present disclosure relates to catheter-based devices and systems for delivering a prosthetic heart valve. More particularly, the present disclosure relates to transcatheter prosthetic heart valve delivery devices and corresponding methods of use.

### BACKGROUND

A human heart includes four heart valves that define the pathway of blood flow through the heart: the mitral valve, the tricuspid valve, the aortic valve, and the pulmonary valve. The mitral and tricuspid valves are atrio-ventricular valves, which are between the atria and the ventricles, while the aortic and pulmonary valves are semilunar valves, which are in the arteries leaving the heart. Ideally, native leaflets of a heart valve move apart from each other when the valve is in an open position and meet or “coapt” when the valve is in a closed position. Problems that may develop with valves include stenosis in which a valve does not open properly, and/or insufficiency or regurgitation in which a valve does not close properly. Stenosis and insufficiency may occur concomitantly in the same valve. The effects of valvular dysfunction vary, with regurgitation or backflow typically having relatively severe physiological consequences to the patient.

Diseased or otherwise deficient heart valves can be repaired or replaced using a variety of different types of heart valve surgeries. One conventional technique involves an open-heart surgical approach that is conducted under general anesthesia, during which the heart is stopped and blood flow is controlled by a heart-lung bypass machine.

More recently, minimally invasive approaches have been developed to facilitate catheter-based implantation of the valve prosthesis on the beating heart, intending to obviate the need for the use of classical sternotomy and cardiopulmonary bypass. In general terms, an expandable prosthetic valve is compressed about or within a catheter, inserted inside a body lumen of the patient, such as the femoral artery, and delivered to a desired location in the heart.

The heart valve prosthesis employed with catheter-based, or transcatheter, procedures generally includes an expandable multi-level frame or stent that supports a valve structure having a plurality of leaflets. The frame can be contracted during percutaneous transluminal delivery and expanded upon deployment at or within the native valve. One type of valve stent can be initially provided in an expanded or uncrimped condition, then crimped or compressed about a balloon portion of a catheter. The balloon is subsequently inflated to expand and deploy the prosthetic heart valve. With other stented prosthetic heart valve designs, the stent frame is formed to be self-expanding. With these systems, the valved stent is crimped down to a desired size and held in that compressed state within a sheath for transluminal delivery. Retracting the sheath from this valved stent allows the stent to self-expand to a larger diameter, fixating at the native valve site. In more general terms, then, once the prosthetic valve is positioned at the treatment site, for instance within an incompetent native valve, the stent frame structure may be expanded to hold the prosthetic valve firmly in place. One example of a stented prosthetic valve is disclosed in U.S. Pat. No. 5,957,949 to Leonhardt et al., which is incorporated by reference herein in its entirety.

The present disclosure addresses problems and limitations associated with the related art.

### SUMMARY

Some aspects of the present disclosure are directed to a system including a prosthetic heart valve and a delivery device. The prosthetic heart valve includes a stent frame and a valve structure. The stent frame is transitionable between a compressed condition and an expanded condition. The valve structure is supported by the stent frame. A first region of the stent frame extends longitudinally beyond the valve structure in a first direction, and a second region of the stent frame extends longitudinally beyond the valve structure in a second direction opposite the first direction. The delivery device includes an outer shaft assembly and an inner shaft assembly. The outer shaft assembly includes a capsule extending distally from an outer shaft. The inner shaft assembly is coaxially received within the outer shaft assembly, and includes an inner shaft, a valve retainer, and a tip unit. The valve retainer is maintained by the inner shaft. The tip unit extends distally from the inner shaft, and defines a proximal section opposite a distal section, and an intermediate section between the proximal and distal sections. An outer diameter of the proximal section is greater than an outer diameter of the inner shaft. An outer diameter of the intermediate section is greater than the outer diameter of the proximal section. An outer diameter of the tip unit increases at a transition from the intermediate section to the distal section. The system is configured to provide a delivery state in which the prosthetic heart valve is contained in the compressed condition over the inner shaft assembly by the capsule, the first region of the stent frame is connected to the valve retainer, and at least a portion of the second region of the stent frame contacts the proximal section of the tip unit.

In some embodiments, the tip unit minimizes or prevents strut crossing and/or infolding along the second region, for example when loading the prosthetic heart valve to the delivery device. As a point of reference, strut crossing or inflow overlap is a non-uniform rearranging of a self-expanding prosthetic heart valve stent frame end crowns under the delivery device's capsule which can occur during initial loading of the prosthetic heart valve. This phenomena can lead to infolding of the stent frame during recapture or deployment. Infolding is a fold in the frame that extends inward away from the anatomy and in a vertical line along the frame. Infolding can have short-term and long-term consequences.

Other aspects of the present disclosure are directed to a system including a prosthetic heart valve and a delivery device. The prosthetic heart valve includes a stent frame and a valve structure. The stent frame is transitionable between a compressed condition and an expanded condition. The valve structure is supported by the stent frame. A first region of the stent frame extends longitudinally beyond the valve structure in a first direction, and a second region of the stent frame extends longitudinally beyond the valve structure in a second direction opposite the first direction. The delivery device includes an outer shaft assembly and an inner shaft assembly. The outer shaft assembly includes a capsule extending distally from an outer shaft. The inner shaft assembly is coaxially received within the outer shaft assembly, and includes an inner shaft, a valve retainer, and a tubular support body. The valve retainer is maintained by the inner shaft. The tubular support body is assembled over the inner shaft and extends distally from the valve retainer. The system is configured to provide a delivery state in which the prosthetic heart valve is contained in the compressed condition over the inner shaft assembly by the capsule, the first region of the stent frame is connected to the valve retainer, and at least a majority of the first region contacts the tubular support body. In some embodiments, the tubular body minimizes or prevents strut crossing and/or infolding along the first region, for example when loading the prosthetic heart valve to the delivery device.

Other aspects of the present disclosure are directed to method of loading a prosthetic heart valve to a delivery device. The prosthetic heart valve includes a stent frame and a valve structure. The stent frame is transitionable between a compressed condition and an expanded condition. The valve structure is supported by the stent frame. A first region of the stent frame extends longitudinally beyond the valve structure in a first direction, and a second region of the stent frame extends longitudinally beyond the valve structure in a second direction opposite the first direction. The method includes receiving a delivery device. The delivery device includes an outer shaft assembly and an inner shaft assembly. The outer shaft assembly includes a capsule extending distally from an outer shaft. The inner shaft assembly is coaxially received within the outer shaft assembly, and includes an inner shaft, a valve retainer, and a tip unit. The valve retainer is maintained by the inner shaft. The tip unit extends distally from the inner shaft, and defines a proximal section opposite a distal section, and an intermediate section between the proximal and distal sections. An outer diameter of the proximal section is greater than an outer diameter of the inner shaft. An outer diameter of the intermediate section is greater than the outer diameter of the proximal section. An outer diameter of tip unit increases at a transition from the intermediate section to the distal section. The method further includes arranging the delivery device and the prosthetic heart valve in a delivery state in which the prosthetic heart valve is contained in the compressed condition over the inner shaft assembly by the capsule, the first region of the stent frame is connected to the valve retainer, and at least a portion of the second region of the stent frame contacts the proximal section. In some embodiments, the step of arranging includes crimping the prosthetic heart valve over the inner shaft assembly. In some embodiments, the second region terminates in a plurality of crowns, and the step of arranging is characterized by the tip unit preventing the crowns from crossing over one another.

### BRIEF DESCRIPTION OF DRAWINGS

FIG. 1A is a side view of a prosthetic heart valve useful with the systems and delivery devices of the present disclosure and in a normal, expanded condition;

FIG. 1B is a side view of the prosthetic heart valve of FIG. 1A in a compressed condition;

FIG. 2 is an exploded, perspective view of a delivery device in accordance with principles of the present disclosure;

FIG. 3 is a simplified illustration of a system in accordance with principles of the present disclosure, including the prosthetic heart valve of FIG. 1A loaded to the delivery device of FIG. 2;

FIG. 4 is an enlarged side view of portions of the delivery device of FIG. 2, including an inner shaft and a tip unit;

FIGS. 5A and 5B illustrate, in simplified form, the prosthetic heart valve of FIG. 1A loaded to the tip unit of FIG. 4;

FIG. 6 is an enlarged side view of another tip unit useful with the delivery device of FIG. 2;

FIG. 7 is a simplified side view of the prosthetic heart valve of FIG. 1B side-by-side with portions of the delivery device of FIG. 2, including a tubular support body and in inner shaft;

FIG. 8 illustrates, in simplified form, portions of the system of FIG. 2 in a delivery state and illustrating an arrangement of the prosthetic heart valve of FIG. 1B relative to the tubular support body of FIG. 7; and

FIGS. 9A and 9B illustrate methods of loading a prosthetic heart valve to a delivery device in accordance with principles of the present disclosure.

### DETAILED DESCRIPTION

Specific embodiments of the present disclosure are now described with reference to the figures, where like reference numbers indicate identical or functionally similar elements.

Aspects of the disclosure are beneficial for use with prosthetic heart valves and heart valve repair methods including the implantation of a prosthetic heart valve, particularly, prosthetic heart valves delivered via a transcatheter procedure. As referred to herein, prosthetic heart valves can include a bioprosthetic heart valve structure having tissue leaflets or a synthetic heart valve having polymeric, metallic, or tissue-engineered leaflets, and can be specifically configured for replacing or repairing valves of the human heart. In one non-limiting example, the valve of the human heart is an aortic valve, although the systems and methods of the present disclosure can be useful with the mitral, tricuspid, or pulmonary heart valve. The prosthetic heart valves of the present disclosure may be self-expandable, balloon expandable and/or mechanically expandable, or combinations thereof. In general terms, the prosthetic heart valves of the present disclosure include a stent or stent frame having an internal lumen maintaining a valve structure (tissue or synthetic), with the stent frame having a normal, expanded condition or arrangement and being collapsible to a compressed condition or arrangement for loading within the delivery device. For example, the stents or stent frames are support structures that include a number of struts or wire segments arranged relative to each other to provide a desired compressibility and strength to the prosthetic valve. The struts or wire segments are arranged such that they are capable of self-transitioning from, or being forced from, a compressed or collapsed arrangement to a normal, radially expanded arrangement. The struts or wire segments can optionally be formed from a shape memory material, such as a nickel titanium alloy (e.g., nitinol). The stent frame can be laser-cut from a single piece of material, or can be assembled from a number of discrete components.

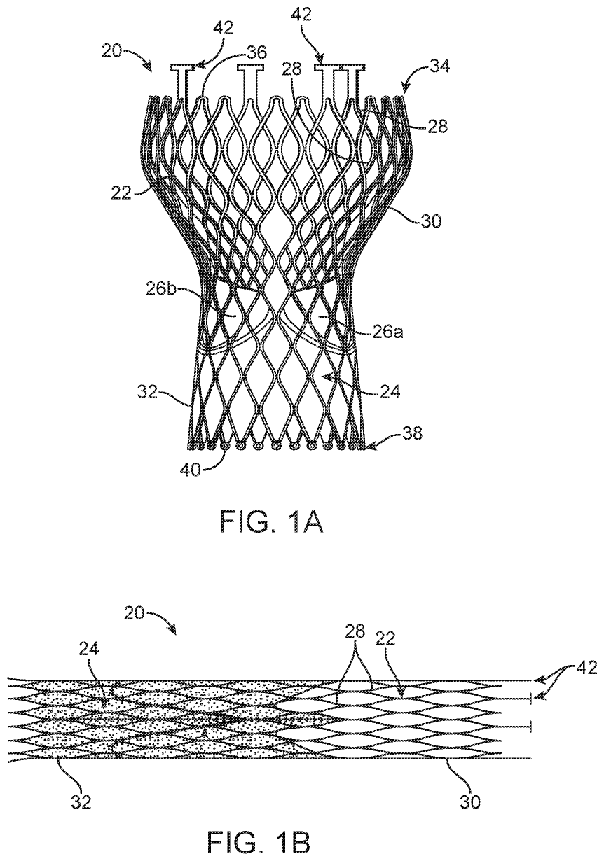

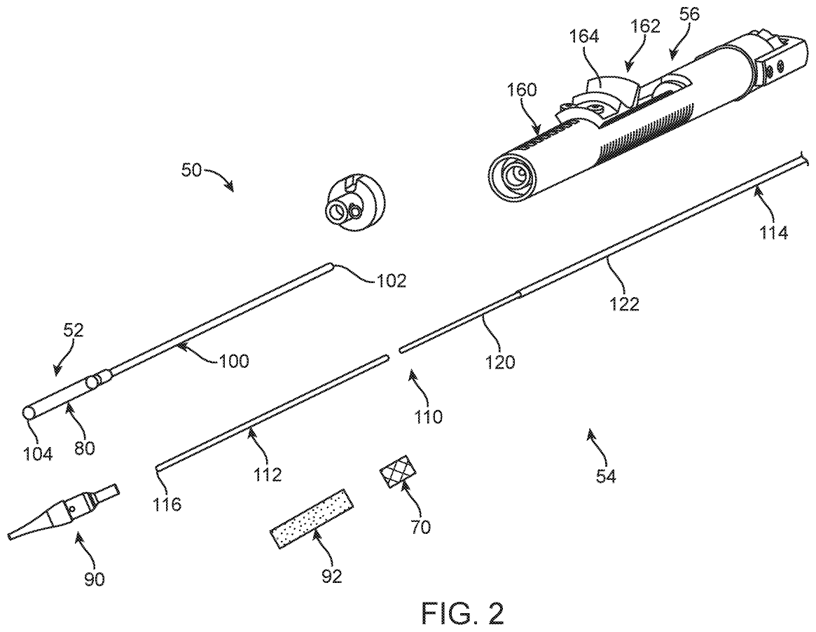

With the above understanding in mind, one non-limiting example of a prosthetic heart valve 20 useful with systems and methods of the present disclosure is illustrated in FIG. 1A. The prosthetic heart valve 20 is shown in a normal or expanded condition in the view of FIG. 1A; FIG. 1B illustrates the prosthetic heart valve 20 in a compressed condition (e.g., when compressively retained within an outer catheter or sheath (not shown)). The prosthetic heart valve 20 includes a stent or stent frame 22 and a valve structure 24. The stent frame 22 can assume any of the forms described above and is generally constructed so as to be self-expandable from the compressed arrangement (FIG. 1B) to the normal, expanded arrangement (FIG. 1A). In other embodiments, the stent frame 22 is expandable to the expanded arrangement by a separate device (e.g., a balloon internally located within the stent frame 22). The valve structure 24 is assembled to the stent frame 22 and provides two or more (typically three) leaflets 26a, 26b. The leaflets 26a, 26b (as well as other portions of the valve structure 24) can be formed from autologous tissue, xenograph material, or synthetics. The leaflets 26a, 26b can be provided as a homogenous, biological valve structure, such as porcine, bovine, or equine valves. Alternatively, the leaflets 26a, 26b can be provided independent of one another (e.g., bovine or equine pericardial leaflets) and subsequently assembled to the stent frame 22. The valve structure 24 can be assembled to the stent frame 22 in various manners, such as by sewing the valve structure 24 to one or more struts or wire segments 28 defined by the stent frame 22. As will be understood by one of ordinary skill, the prosthetic heart valve 20 can include additional components conventionally employed, such as a skirt or similar structure.

With the example construction of FIGS. 1A and 1B, the prosthetic heart valve 20 is configured for replacing an aortic valve. Alternatively, other shapes are also envisioned, adapted for the specific anatomy of the valve to be replaced (e.g., prosthetic heart valves in accordance with the present disclosure can alternatively be shaped and/or sized for replacing a native mitral, pulmonic, or tricuspid valve). Regardless, the valve structure 24 can be arranged to extend less than an entire length of the stent frame 22 to define a first region 30 opposite a second region 32. The first region 30 extends to a first end 34 of the stent frame 22, with crowns 36 being formed at an intersection of two (or more) adjacent ones of the wire segments 28. Similarly, the second region 32 extends to a second end 38 of the stent frame 22 at which crowns 40 are formed. In some embodiments, the first and second regions 30, 32 can be viewed as being free of, or not including, the leaflets 26a, 26b or other delicate structures of the prosthetic heart valve 20.

A configuration and arrangement of the leaflets 26a, 26b defines a direction of blood flow through the prosthetic heart valve 20, with the first region 30 serving as an outflow region or outflow side, and the second region 32 serving as an inflow region or inflow side. The terms “inflow” and “outflow” can also be in reference to an arrangement of the prosthetic heart valve 20 upon final implantation relative to the native aortic valve (or other valve) being replaced. With embodiments in which the prosthetic heart valve 20 is to be implanted via a retrograde approach, the prosthetic heart valve 20 will be arranged within the corresponding delivery device such that the inflow region 30 is proximal the outflow region 32. With these conventions in mind, one or more connector bodies 42 (e.g., posts, tabs, etc.) are optionally formed by, or provided with, the stent frame 22 at the first end 34 (i.e., the first or outflow region 30), for example as extensions from a corresponding one of the crowns 36. The connector bodies 42 (e.g., posts, tabs, etc.) are adapted for connection to a corresponding component(s) of the delivery device as described below. While the stent frame 22 is shown in FIGS. 1A and 1B as having four of the connector bodies 42, any other number, either greater or lesser, is equally acceptable. For example, the stent frame 22 can include as few as a single one of the connector bodies 42. In yet other embodiments, the connector bodies 42 can be omitted.

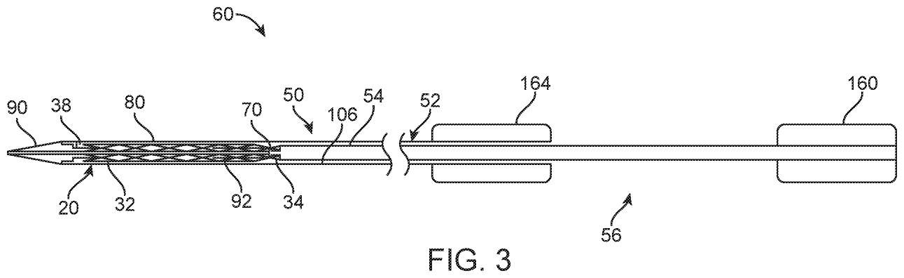

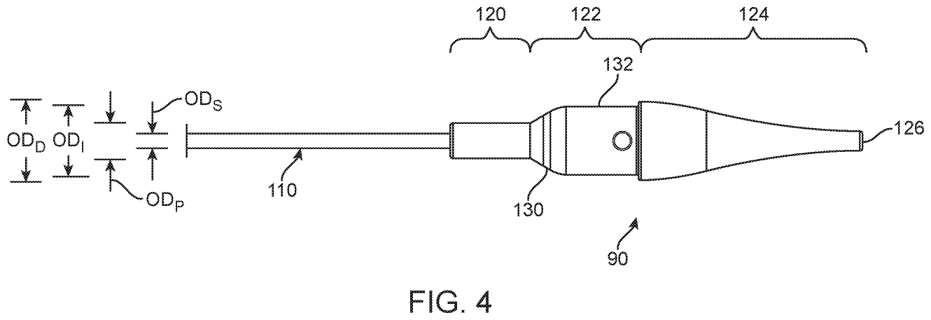

With the above understanding of the prosthetic heart valve 20 in mind, one embodiment of a delivery device 50 in accordance with principles of the present disclosure is shown in FIG. 2. The delivery device 50 includes an outer shaft assembly 52, an inner shaft assembly 54, and a handle 56. With additional reference to FIG. 3, the delivery device 50 combines with the prosthetic heart valve 20 to form a system 60 in accordance with principles of the present disclosure for restoring (e.g., replacing) a defective heart valve of a patient. The delivery device 50 is generally configured for percutaneously delivering the prosthetic heart valve 20, in the compressed condition, to a target site and deploying the prosthetic heart valve 20 at the target site. For example, the delivery device 50 provides a delivery state (FIG. 3) in which the prosthetic heart valve 20 is coupled to a valve retainer 70 (schematically illustrated in FIG. 2) of the inner shaft assembly 54, and compressively retained within a capsule 80 of the outer shaft assembly 52. The outer shaft assembly 52 can be manipulated to withdraw the capsule 80 proximally from the prosthetic heart valve 20 via operation of the handle assembly 56 (as schematically reflected by FIG. 3) in defining a deployment state of the delivery device 50. In the deployment state, withdrawing the capsule 80 permits the prosthesis 20 to self-expand (alternatively, be caused to expand) and release from the inner shaft assembly 54. Other features discussed below, where provided, can operate to effectuate this release. Regardless, the inner shaft assembly 54 includes one or more features described below, such as a tip unit 90 and/or a tubular support body 92, that support the prosthetic heart valve 20 during loading onto the inner shaft assembly 54 and while in the delivery state. The tip unit 90 and/or the tubular support body 92 minimizes or prevents crossing or infolding of the stent frame 22 at one or both of the first and second regions 30, 32.

Various features of the components 52-56 reflected in FIGS. 2 and 3 and described below can be modified or replaced with differing structures or mechanisms. Thus, the present disclosure is in no way limited to the outer shaft assembly 52, the inner shaft assembly 54, the handle assembly 56, etc., as shown and described below. More generally, then, some delivery devices in accordance with principles of the present disclosure provide features capable of retaining a self-deploying stented prosthetic heart valve (e.g., the capsule 80), along with one or more components (e.g., the tip unit 90 and/or the tubular support body 92) capable of supporting the prosthetic heart valve when compressed or crimped over the inner shaft assembly 54.

In some embodiments, the outer shaft assembly 52 includes the capsule 80 and a shaft 100, and defines a proximal end 102 and distal end 104. A lumen 106 (identified in FIG. 3) is formed by the outer shaft assembly 52, extending from the distal end 104 through the capsule 80 and at least a portion of the shaft 100. The lumen 106 can be open at the proximal end 102. The capsule 80 extends distally from the shaft 100, and in some embodiments has a more stiffened construction (as compared to a stiffness of the shaft 80) that exhibits sufficient radial or circumferential rigidity to overtly resist the expected expansive forces of the prosthetic heart valve 20 when compressed within the capsule 80. For example, the shaft 100 can be a polymer tube embedded with a metal braiding, whereas the capsule 80 may include a laser-cut metal tube that is optionally embedded within a polymer covering. Alternatively, the capsule 80 and the shaft 100 can have a more uniform construction (e.g., a continuous polymer tube). Regardless, the capsule 80 is constructed to compressively retain the stented prosthetic heart valve 20 at a predetermined diameter when loaded within the capsule 80, and the shaft 100 serves to connect the capsule 80 with the handle assembly 56. The shaft 100 (as well as the capsule 80) is constructed to be sufficiently flexible for passage through a patient's vasculature, yet exhibits sufficient longitudinal rigidity to effectuate desired axial movement of the capsule 80. In other words, proximal retraction of the shaft 100 is directly transferred to the capsule 80 and causes a corresponding proximal retraction of the capsule 80. In other embodiments, the shaft 100 is further configured to transmit a user-generated rotational force or movement onto the capsule 80.

The inner shaft assembly 54 can have various constructions for supporting the prosthetic heart valve 20. The inner shaft assembly 54 is sized and shaped to extend within the central lumen 106 of the outer shaft assembly 52. The inner shaft assembly 54 includes an inner shaft 110, the valve retainer 70, a distal tip (for example provided by the optional tip unit 90), and the optional tubular support body 92. The inner shaft 110 is sized to be slidably received within the lumen 106 of the outer shaft assembly 52 and is configured for mounting of the valve retainer 70, the tip unit 90 and the tubular support body 92 (where provided). As shown in FIG. 2, the inner shaft 110 can include a distal segment 112 and a proximal segment 114. The distal segment 112 terminates at a distal end 116 and connects the tip unit 90 to the proximal segment 114. The proximal segment 114, in turn, couples the inner shaft assembly 54 to the handle assembly 56. The inner shaft 110 can define a continuous lumen (hidden) sized to slidably receive an auxiliary component such as a guide wire (not shown).

The distal segment 112 can be a flexible polymer tube embedded with a metal braid. Other constructions are also acceptable so long as the distal segment 112 exhibits sufficient structural integrity to support a loaded, compressed prosthetic heart valve. The proximal segment 114 can include, in some constructions, a leading portion 120 and a trailing portion 122. The leading portion 120 serves as a transition between the distal and proximal segments 112, 114, and thus in some embodiments is a flexible polymer tubing (e.g., PEEK) having an outer diameter slightly less than that of the distal segment 112. The trailing portion 122 can have a more rigid construction (e.g., a metal hypotube), adapted for robust assembly with the handle assembly 56. Other materials and constructions are also envisioned. For example, in alternative embodiments, the distal and proximal segments 112, 114 are integrally formed as a single, homogenous tube or solid shaft.

The valve retainer 70 can assume various forms appropriate for selectively capturing or connecting to a corresponding feature of the prosthetic heart valve 20. With additional reference to FIGS. 1A and 1B, with some embodiments in which the prosthetic heart valve 20 includes the connector bodies 42, the valve retainer 70 can include or define slots sized and shaped to receive respective ones of the connector bodies 42. For example, the valve retainer 70 can be a hub or spindle into which slot(s) are formed. Other constructions are equally acceptable. Further, the inner shaft assembly 54 can optionally include additional components or mechanisms that assist in temporarily securing a corresponding feature of the prosthetic heart valve 20 to the valve retainer 70 and/or releasing the prosthetic heart valve 20 from the valve retainer 70 upon full retraction of the capsule 80. Regardless, in the delivery state, a length of the prosthetic heart valve 20 (in the collapsed condition) is effectively predetermined. Thus, because a location of an end of the prosthetic heart valve 20 as secured to the valve retainer 70 is predetermined, a location of the opposite end of the prosthetic heart valve 20 relative to the delivery device 50 is also effectively predetermined. For example, in FIG. 3, the first end 34 is secured to valve retainer 70; with the prosthetic heart valve 20 in the collapsed condition, a longitudinal location of the second region 32, and in particular the second end 38, relative to the other components of the delivery device 50 (e.g., relative to the tip unit 90) is also essentially predetermined. In some embodiments, the tip unit 90 incorporates features configured to support to the second region 32 at or near this essentially predetermined location as described below.

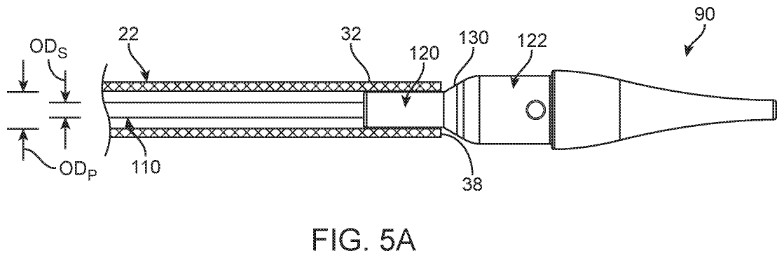

With the above in mind, one embodiment of the tip unit 90 is shown in greater detail in FIG. 4. As reflected by FIG. 4, upon final assembly, the tip unit 90 extends distally from the inner shaft 110. A shape or profile of the tip unit 90 defines a proximal section 120, an intermediate section 122, and a distal section 124. The intermediate section 122 extends between the proximal and distal sections 120, 124. An outer diameter OD_{P} of the proximal section 120 is greater than an outer diameter OD_{S} of the inner shaft 110 (at least along the section of the inner shaft 90 immediately proximate the tip unit 90). An outer diameter OD_{I} of the intermediate section 122 is greater than the outer diameter OD_{P} of the proximal section 120. An outer diameter of the tip unit 90 increases at a transition from the intermediate section 122 to the distal section 124, for example to a maximum distal section outer diameter OD_{D}. Finally, the distal section 124 can define a distally tapering outer profile in extension from the intermediate section 122 to a tip end 126, adapted, for example, to promote atraumatic contact with bodily tissue.

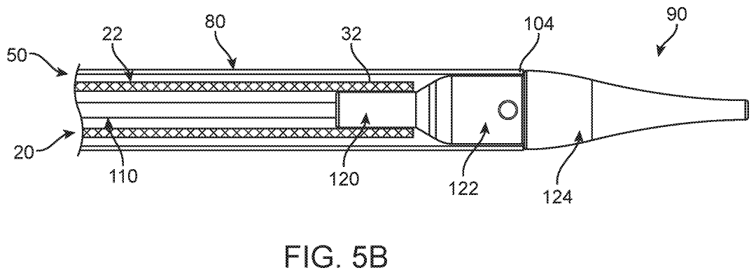

In some embodiments, the outer diameter OD_{P} of the proximal section 120 is selected to support a corresponding region of the prosthetic heart valve 20 (FIGS. 1A and 1B) during and following crimping of the prosthetic heart valve 20 over the inner shaft 110, for example to minimize a propensity of the prosthetic heart valve 20 to experience strut crossing or overlap. For example, FIG. 5A shows a simplified representation of a portion of the stent frame 22 having been crimped to the collapsed condition over the inner shaft 110 (i.e., the delivery state). For pictorial simplicity, the capsule 80 (FIG. 2) is omitted from the view of FIG. 3, with the understanding that the capsule 80 constrains the stent frame 22 in the collapsed condition in the delivery state (FIG. 3). It will be recalled that in the delivery state, a location of the second region 32 of the stent frame 22 relative to the tip unit 90 is essentially predetermined. With this in mind, the tip unit 90 is located so as to position the proximal section 120 to be radially aligned with or radially disposed inside of at least a portion of the second region 32 immediately proximate the second end 38. While FIG. 5A illustrates the second end 38 as aligned with the proximal section 120, in other embodiments, the second end 38 may be located along or aligned with the intermediate section 122.

FIG. 5A generally reflects that in the delivery state, a portion of the second region 32 is crimped or collapsed into contact with a surface of the proximal section 120 of the tip unit 90. In other arrangements, a slight radial gap may exists between the second region 32 and a surface of the proximal section 120. Regardless, a portion of the second region 30 proximate the second end 38 is aligned with the proximal section 120, with the proximal section 120 providing a surface for supporting the second region 32 (e.g., the struts 28 and the crowns 40 (FIGS. 1A and 1B) of the stent frame 22) during and following crimping of the prosthetic heart valve 20 over the inner shaft 110 due, for example, to the outer diameter OD_{P} of the proximal section 120 being greater that the outer diameter OD_{S} of the inner shaft 110. In other words, absent the proximal section 120, the second end 38 and other portions of the second region 32 could freely deflect radially inward toward the inner shaft 110 in a manner that may otherwise have a propensity to allow the struts 28 and/or the crowns 40 along the second region 32 to undesirably overlap or cross. Support provided by the proximal section 120 minimizes or prevents strut crossing or overlap from occurring along the second region 32 proximate the first end 38.

The tip unit 90 can include or exhibit additional, beneficial profile features. For example, and with additional reference to FIG. 4, in some embodiments the intermediate section 122 can be shaped to define a trailing segment 130 and a leading segment 132. The trailing segment 130 extends from the proximal section 120 to the leading segment 132, and the leading segment 132 extends from the trailing segment 130 to the distal section 124. With these definitions in mind, in some embodiments the trailing segment 130 defines a smooth, continuously expanding outer diameter from the proximal section 120 to the leading segment 132. With this smooth, continuously expanding diameter profile or contour, the second end 38 can readily slide along the trailing segment 130 (as compared to a shape in which a more abrupt edge or shoulder were formed at the transition from the proximal section 120 to the intermediate section 122). Thus, when the delivery device 50 (FIG. 2) is manipulated through tortuous anatomy, the trailing segment 130 facilitates or allows for slight movements of the second end 38 so as to minimize possible damage to the stent frame 22.

The leading segment 132 can have a substantially uniform outer diameter (i.e., within 5 percent of a truly uniform outer diameter) in extension from the trailing segment 130 to the distal section 124. With these and other constructions, the intermediate section 122 is configured to receive and support the capsule 80 as schematically illustrated in FIG. 5B. As shown, in the delivery state, the distal end 104 of the capsule 80 can be located along the intermediate section 122 immediately adjacent the distal section 124. In some embodiments, the maximum distal section outer diameter OD_{D} (FIG. 4) of the distal section 124 can approximate the outer diameter of the capsule 80 at the distal end 104. Regardless, FIG. 5B reflects that the proximal section 120 effectively increases a packing density inside the capsule 80 (as compared to a packing density were the proximal segment 120 not present). This increased packing density serves to minimize or prevent the struts 28 and the crowns 40 (FIGS. 1A and 1B) of the second region 32 of the stent frame 22 from folding over one another when loading the prosthetic heart valve 20 to the delivery device 50 (as well as after the prosthetic heart valve 20 has been loaded).

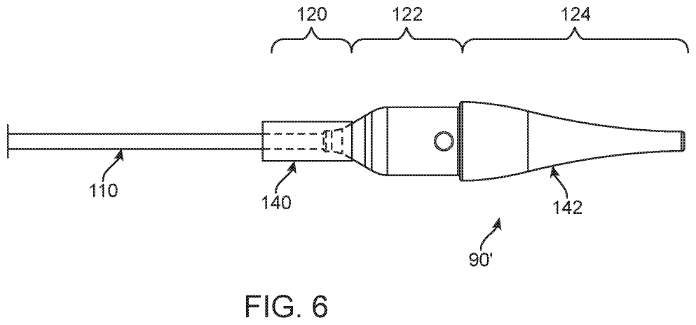

In the embodiments shown and described, the tip unit 90 is formed as a homogenous, integral body (e.g., a molded polymeric body). In other embodiments, the tip unit 90 can be formed or defined by two (or more) separately formed bodies. For example, FIG. 6 illustrates another embodiment tip unit 90′ assembled to the inner shaft 110. The tip unit 90′ includes a first or support body 140 and a second or tip body 142. The first and second bodies 140, 142 are separately formed and assembled to the inner shaft 110 in various manners. For example, the first and second bodies 140, 142 can be secured to one another by threading, compression, snap fit, over molding, etc. In other embodiments, the first and second bodies 140, 142 need not necessarily be physically secured or attached to one another, and can be arranged in an abutting relationship upon assembly to the inner shaft 110. In some embodiments, the first body 140 is configured and arranged to define the proximal section 120 as described above, whereas the second body 142 is configured and arranged to define the intermediate and distal sections 122, 124. The first and second bodies 140, 142 can be formed of the same or differing materials. For example, the second body 142 can be a molded polymer conventionally employed with transcatheter prosthetic heart valve delivery devices. The first body 140 can be formed of a more resilient material (e.g., a soft or resilient polymer ring or tube). Regardless, the tip unit 90′ provides the same or similar features as the tip unit 90 (FIG. 5A) above, including the proximal section 120 minimizing or preventing strut crossing or overlap during (and following) loading of the prosthetic heart valve 20 (FIG. 1A).

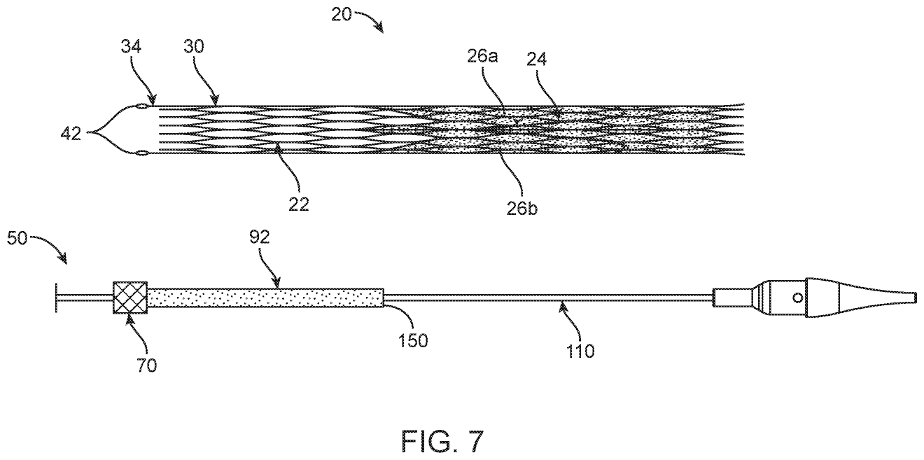

Returning to FIGS. 2 and 3, where provided, the tubular support body 92 can assist in minimizing or preventing infolding or crossing at the first end 34 of the stent frame 22. With reference to FIG. 7, the tubular support body 92 can be formed of a material appropriate for contact with the prosthetic heart valve 20. As shown, the tubular support body 92 is assembled over the inner shaft 110 adjacent the valve retainer 70 and extends distally from the valve retainer 70. An outer diameter of the tubular support body 92 is greater than an outer diameter of the inner shaft 110, with the tubular support body 92 thus providing a surface for supporting the prosthetic heart valve 20 during and after loading as described below. In some embodiments, a length of the tubular support body 92, or distal extension of the tubular support body 92 relative to the valve retainer 70, is selected in accordance with the expected location of one or more components of the prosthetic heart valve 20 when loaded to the delivery device 20 while maximizing a length of the support provided to the stent frame 22.

For example, upon final assembly, the tubular support body 92 terminates at a distal side 150 opposite the valve retainer 70. A length and location of the tubular support body 92 is selected such that a distal side 150 of the tubular support body 92 is proximally spaced from delicate structures of the prosthetic heart valve 20 during loading, for example the valve structure 24 and in particular the leaflets 26a, 26b. With this construction, the leaflets 26a, 26b will not contact the tubular support body 92 during loading in a manner that might otherwise damage the leaflets 26a, 26b. By way of further explanation, it will be recalled that in the delivery state, the first end 34 of the stent frame 22 is connected to the valve retainer 70, for example via the connector bodies 42. A longitudinal distance from the connector bodies 42 to the leaflets 26a, 26b in the collapsed condition is thus essentially predetermined. A length of the tubular support body 92 and/or a longitudinal distance from the valve retainer 70 to the distal side 150 can be selected as a function of this essentially predetermined dimension of the prosthetic heart valve 20. FIG. 7 illustrates the prosthetic heart valve 20 in the collapsed condition apart from the delivery device 50, but in alignment with a longitudinal location when loaded over the inner shaft 110. With this in mind, the tubular support body 92 is configured and arranged to so as to locate the distal side 150 proximal the leaflets 26a, 26b by a sufficient distance to ensure that the leaflets 26a, 26b (as well as, perhaps, other portions of the valve structure 24 and/or other delicate surfaces of the prosthetic heart valve 20) will not contact the tubular support body 92 during and following loading. Apart from this design parameter, the tubular support body 92 can extend well beyond the valve retainer 70 so as to provide substantive support for the stent frame 22.

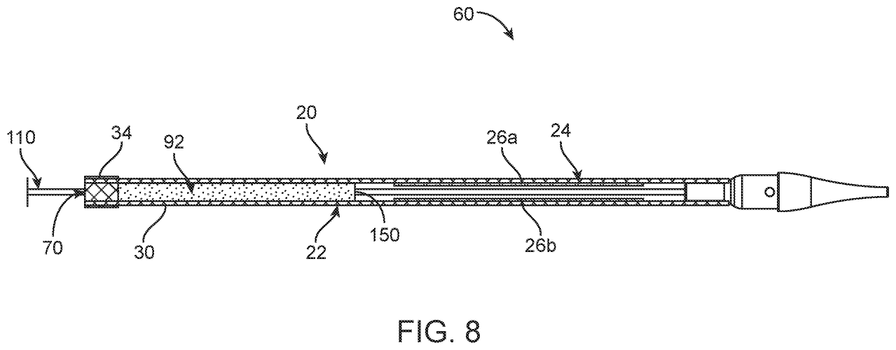

One acceptable arrangement of the tubular support body 92 relative to the prosthetic heart valve 20 in the delivery state is shown in simplified form in FIG. 8 (for pictorial simplicity, the outer shaft assembly 52 (FIG. 2) is omitted from the view). The first end 34 of the stent frame 22 is connected to the valve retainer 70. A portion of the stent frame 22 along the first region 30 is crimped or collapsed into contact with a surface of the tubular support body 92. In other arrangements, a slight radial gap may exists between the stent frame 22 and the tubular support body 92. Regardless, a portion of the first region 30 proximate the first end 34 is aligned with or radially disposed outside of the tubular support body 92, with the tubular support body 92 providing a surface for supporting the first region 30 (e.g., the struts 28 and the crowns 36 (FIGS. 1A and 1B) of the stent frame 22) during and following crimping of the prosthetic heart valve 20 over the inner shaft 110 due, for example, to the outer diameter of the tubular support body 92 being greater that the outer diameter of the inner shaft 110. In other words, absent the tubular support body 92, portions of the stent frame 22 along the first region 30 could freely deflect radially inward toward the inner shaft 110 in a manner that may otherwise have a propensity to allow the struts 28 along the first region 30 to undesirably overlap or cross. Support provided by the tubular support body 92 minimizes or prevents strut crossing or overlap from occurring along the first region 30. The distal side 150 is proximally spaced from the leaflets 26a, 26b (and in some embodiments, from other portions of the valve structure 24), such that the leaflets 26a, 26b do not contact the tubular support body 92. In some embodiments, however, the tubular support body 92 is sized and located to extend along and potentially interface with a substantial length of the first region 30. For example, in some embodiments, the tubular support body 92 is radially aligned with or disposed radially inside of at least a majority of a length of the first region 30 (e.g., a length of the tubular support body 92 is at least 50 percent of a longitudinal length from the first end 34 to the leaflets 26a, 26b of the prosthetic heart valve 20 in the compressed condition).

Returning to FIG. 2, the handle assembly 56 can assume various forms appropriate for user handling and operation of the delivery device 50. In some embodiments, the handle assembly 56 includes a housing 160 and one or more actuator mechanisms 162 (referenced generally). The housing 160 generally provides a surface for convenient handling and grasping by a user, and may have the generally cylindrical shape as shown, although other shapes and sizes are also acceptable. The housing 160 maintains the actuator mechanism 162, with the handle assembly 56 configured to facilitate sliding movement of the outer shaft assembly 52 relative to the inner shaft assembly 54 (and/or vice-versa). In one simplified construction of the actuator mechanism 162, a user interface or actuator 164 is slidably retained by the housing 160 and is coupled to the proximal end 102 of the outer shaft assembly 52. A proximal end of the inner shaft assembly 54 is secured to the housing 160. With this optional construction, sliding movement of the actuator 164 co-axially advances/retracts the outer shaft assembly 52 relative to the inner shaft assembly 54 as illustrated by the schematic representation of the handle assembly 56 in FIG. 3. Although shown as a slide mechanism, other constructions and/or devices may be used to retrace/advance the outer shaft assembly 52 relative to the inner shaft assembly 54 (and/or vice-versa), such as, but not limited to, rotating mechanisms, sliding mechanisms that are coaxially disposed over the inner shaft assembly 54, combinations of rotating and sliding mechanisms, and other advancement/retraction mechanisms apparent to those of ordinary skill in the art. The handle assembly 56 can optionally include one or more additional components or mechanisms.

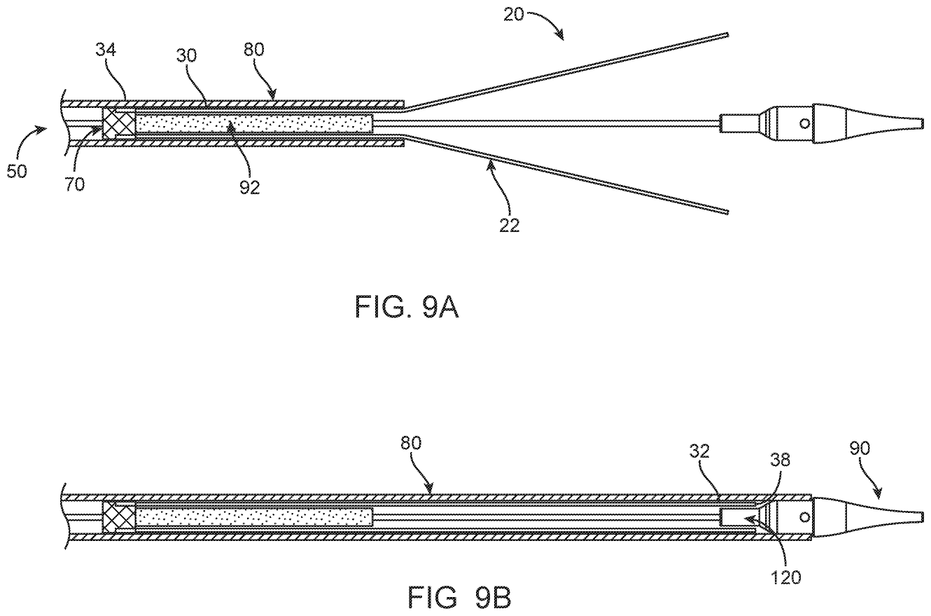

The prosthetic heart valve 20 can be loaded to the delivery device 50 in various fashions and optionally with the assistance of one or more tools. In general terms, with the outer shaft assembly 52 retracted relative to the inner shaft assembly 54 so as to locate the capsule 80 proximal the valve retainer 70, the prosthetic heart valve 20 is arranged over the inner shaft 110. In this initial stage, portions of the prosthetic heart valve 20 may or may not be partially or fully crimped toward the collapsed condition. Regardless, and with reference to FIG. 9A, the first end 34 of the stent frame 22 is connected to the valve retainer 70, for example via the connector bodies 42 (FIGS. 1A and 1B). The capsule 80 is distally advanced over the prosthetic heart valve 20, with the portion of the prosthetic heart valve 20 within the capsule 80 being crimped and maintained in the collapsed condition by the capsule. At the stage of loading represented by FIG. 9A, for example, the capsule 80 has been incrementally advanced to encompass the first region 30 such that the first region 30 is maintained in the collapsed condition; the portion of the prosthetic heart valve 20 distal the capsule 80 may or may not be partially collapsed or crimped. Regardless, with optional embodiments in which the delivery device 50 includes the tubular support body 92, as the first region 30 is crimped and contained within the capsule 80, at least some of the struts 28 and/or crowns 36 (FIGS. 1A and 1B) along the first region 30 (e.g., proximate the first end 34) may be directed radially inwardly and into contact with the tubular support body 92; under these circumstances, presence of the tubular support body 92 minimizes opportunities for, and in some embodiments prevents, the so-directed struts 28 and/or crowns 36 from infolding and/or crossing over one another.

FIG. 9B illustrates a later stage of the loading process. The capsule 80 has been further distally advanced to encompass the second region 32 such that the second region 32 is maintained in the collapsed condition. As the second region 32 is crimped and contained within the capsule 80, at least some of the struts 28 and/or crowns 40 (FIGS. 1A and 1B) along the second region 32 (e.g., proximate the second end 38) may be directed radially inwardly and into contact with the proximal section 120 of the tip unit 90; under these circumstances, presence of the proximal section 120 minimizes opportunities for, and in some embodiments prevents, the so-directed struts 28 and/or crowns 40 from infolding or crossing over one another.

The transcatheter prosthetic heart valve delivery devices and systems of the present disclosure provide a marked improvement over previous designs. As compared to conventional designs, the delivery devices of the present disclosure provide additional support to one or both end regions of the prosthetic heart valve during and after loading, thus minimizing or preventing strut and/or crown crossing or infolding.

Although the present disclosure has been described with reference to preferred embodiments, persons skilled in the art will recognize that changes can be made in form and detail without departing from the spirit and scope of the present disclosure.

What is claimed is:

1. A system comprising:
a prosthetic heart valve including:
a stent frame transitionable between a compressed condition and an expanded condition,
a valve structure supported by the stent frame,
wherein a first region of the stent frame extends longitudinally beyond the valve structure in a first direction, and a second region of the stent frame extends longitudinally beyond the valve structure in a second direction opposite the first direction; and
a delivery device including:
an outer shaft assembly including a capsule extending distally from an outer shaft,
an inner shaft assembly coaxially received within the outer shaft assembly, the inner shaft assembly including:
an inner shaft,
a valve retainer maintained by the inner shaft,
a tip unit extending distally from the inner shaft, the tip unit defining a proximal section opposite a distal section, and an intermediate section between the proximal section and the distal section,
wherein:
an outer diameter of the proximal section is greater than an outer diameter of the inner shaft,
an outer diameter of the intermediate section is greater than the outer diameter of the proximal section,
an outer diameter of the tip unit increases at a transition from the intermediate section to the distal section;
wherein the system is configured to provide a delivery state in which the prosthetic heart valve is contained in the compressed condition over the inner shaft assembly by the capsule, the first region of the stent frame is connected to the valve retainer, and at least a portion of the second region of the stent frame contacts the proximal section.

2. The system of claim 1, wherein the capsule terminates in a distal end, and further wherein the delivery state includes the distal end disposed over the intermediate section.

3. The system of claim 1, wherein the distal section defines a tapering outer diameter extending from the intermediate section to a distal end of the tip unit.

4. The system of claim 1, wherein the intermediate section defines a trailing segment extending from the proximal section and a leading segment extending from the trailing segment to the distal section, and further wherein the trailing segment defines an expanding outer diameter from the proximal section to the leading segment.

5. The system of claim 4, wherein the leading segment defines a substantially uniform diameter extending from the trailing segment to the distal section.

6. The system of claim 1, wherein the tip unit is an integral, homogenous body secured to the inner shaft.

7. The system of claim 1, wherein the tip unit includes:
a tip body defining the intermediate and distal sections; and
a support body provided apart from the tip body and defining the proximal section.

8. The system of claim 7, wherein the tip body and the support body are manufactured from different materials.

9. The system of claim 1, wherein the inner shaft assembly further includes a tubular support body assembled over the inner shaft and extending distally from the valve retainer.

10. The system of claim 9, wherein the delivery state further includes at least a portion of the first region of the stent frame in contact with the tubular support body.

11. A system comprising:
a prosthetic heart valve including:
a stent frame transitionable between a compressed condition and an expanded condition,
a valve structure supported by the stent frame,
wherein a first region of the stent frame extends longitudinally beyond the valve structure in a first direction, and a second region of the stent frame extends longitudinally beyond the valve structure in a second direction opposite the first direction; and
a delivery device including:
an outer shaft assembly including a capsule extending distally from an outer shaft,
an inner shaft assembly coaxially received within the outer shaft assembly, the inner shaft assembly including:
an inner shaft,
a valve retainer maintained by the inner shaft,
a tubular support body assembled over the inner shaft and extending distally from the valve retainer;
wherein the system is configured to provide a delivery state in which the prosthetic heart valve is contained in the compressed condition over the inner shaft assembly by the capsule, the first region of the stent frame is connected to the valve retainer, and at least a majority of the first region contacts the tubular support body.

12. The system of claim 11, wherein the inner shaft assembly further includes a tip unit extending distally from the inner shaft, the tip unit defining a proximal section opposite a distal section, and an intermediate section between the proximal and distal sections.

13. The system of claim 12, wherein the delivery state further includes at least a portion of the second region in contact with the proximal section.

14. The system of claim 13, wherein:
an outer diameter of the proximal section is greater than an outer diameter of the inner shaft;
an outer diameter of the intermediate section is greater than the outer diameter of the proximal section; and
an outer diameter of tip unit increases at a transition from the intermediate section to the distal section.

15. The system of claim 11, wherein the valve structure is configured and arranged relative to the stent frame such that the first region is an outflow region and the second region is an inflow region.

16. A method of loading a prosthetic heart valve to a delivery device, the prosthetic heart valve including a stent frame transitionable between a compressed condition and an expanded condition, and a valve structure supported by the stent frame, wherein a first region of the stent frame extends longitudinally beyond the valve structure in a first direction, and a second region of the stent frame extends longitudinally beyond the valve structure in a second direction opposite the first direction, the method comprising:
receiving a delivery device including:
an outer shaft assembly including a capsule extending distally from an outer shaft,
an inner shaft assembly coaxially received within the outer shaft assembly, the inner shaft assembly including:
an inner shaft,
a valve retainer maintained by the inner shaft,
a tip unit extending distally from the inner shaft, the tip unit defining a proximal section opposite a distal section, and an intermediate section between the proximal and distal sections,
wherein:
an outer diameter of the proximal section is greater than an outer diameter of the inner shaft,
an outer diameter of the intermediate section is greater than the outer diameter of the proximal section,
an outer diameter of tip increases at a transition from the intermediate section to the distal section; and
arranging the delivery device and the prosthetic heart valve in a delivery state in which the prosthetic heart valve is contained in the compressed condition over the inner shaft assembly by the capsule, the first region of the stent frame is connected to the valve retainer, and at least a portion of the second region of the stent frame contacts the proximal section.

17. The method of claim 16, wherein the step of arranging includes crimping the prosthetic heart valve over the inner shaft assembly.

18. The method of claim 17, wherein the second region terminates in a plurality of crowns, and further wherein the step of arranging is characterized by the tip unit preventing the crowns from crossing over one another.

19. The method of claim 16, wherein the inner shaft assembly further includes a tubular support body assembled over the inner shaft and extending distally from the valve retainer, and further wherein the delivery state includes at least a portion of the first region of the stent frame in contact with the tubular support body.

20. The method of claim 19, wherein the first region terminates in a plurality of crowns, and further wherein the step of arranging is characterized by the tubular support body preventing the crowns from crossing over one another.
